(19) United States
(12) Patent Application Publication
(10) Pub. No.: US 2025/0302475 A1
(43) Pub. Date: Oct. 2, 2025

(54) MULTIPLE HEMOSTASIS CLIP SYSTEM

(71) Applicant: BOSTON SCIENTIFIC SCIMED, INC., Maple Grove, MN (US)

(72) Inventors: Austin Grant JOHNSON, Worcester, MA (US); Colby HARRIS, Norfolk, MA (US); Gonzalo Jose SAENZ VILLALOBOS, Alajuela (CR); Barry WEITZNER, Acton, MA (US)

(21) Appl. No.: 19/065,529

(22) Filed: Feb. 27, 2025

Related U.S. Application Data
(60) Provisional application No. 63/573,201, filed on Apr. 2, 2024.

(51) Int. Cl. A61B 17/08; A61B 17/00

(52) U.S. Cl. CPC A61B 17/083; A61B 17/00234; A61B 2017/00296; A61B 2017/00367; A61B 2017/00477

(57) ABSTRACT

A system includes first and second clips. The first clip includes a first capsule having a first channel and first clip arms, proximal ends of which are slidably received within the first channel to move the first arms between (a) first distal ends of the first arms are separated to receive tissue and (b) the first ends are drawn to grip tissue. The second clip includes a second capsule having a second channel and second clip arms, proximal ends of which are slidably received within the second channel to move the second arms between (a) second distal ends of the second arms are separated to receive tissue and (b) the second ends are drawn to grip tissue. The second ends engage a pivot feature of the first clip so that the first and second clips are releasably and pivotally connected to one another in an insertion configuration.

### PRIORITY CLAIM

The present disclosure claims priority to U.S. Provisional Patent Application Ser. No. 63/573,201 filed Apr. 2, 2024; the disclosure of which is incorporated herewith by reference

### FIELD

present disclosure relates to endoscopic devices and, in particular, relates to endoscopic clipping devices for treating tissue along the gastrointestinal tract.

### BACKGROUND

Physicians have become more willing to perform aggressive interventional and therapeutic endoscopic gastrointestinal (GI) procedures, which may increase the risk of perforating the wall of the GI tract or may require closure of the GI tract wall as part of the procedure. Such procedures may include, for example, the removal of large lesions, tunneling under the mucosal layer of the GI tract to treat issues below the mucosa, full thickness removal of tissue, treatment of issues on other organs by passing outside of the GI tract, and endoscopic treatment/repair of post-surgical issues (e.g., post-surgical leaks, breakdown of surgical staple lines, and anastomotic leaks).

Tissue may be treated via endoscope closure devices such as, for example, hemostasis clips inserted through an endoscope. In some cases, however, multiple clips are required to stop a bleed and/or close a tissue defect. In general, in these cases, each of the clips are individually passed through the endoscope to treat the target area. In addition, each individual clip is inserted to the target area via a single use catheter which must be disposed of upon deployment of each clip.

### SUMMARY

The present disclosure relates to a clipping system for treating tissue which includes first and second clips. The first clip includes a first capsule extending longitudinally from a proximal end to a distal end and including a first channel extending therethrough, and a first pair of clip arms, proximal ends of which are slidably received within the first channel to move the first clip arms between an open configuration, in which distal ends of the first clip arms are separated from one another to receive a tissue therebetween, and a closed configuration, in which the distal ends of the first clip arms are drawn toward one another to grip a tissue therebetween. The proximal end of the first capsule includes a pivot feature extending across the first channel thereof.

The second clip includes a second capsule extending longitudinally from a proximal end to a distal end and including a second channel extending therethrough, and a second pair of clip arms, proximal ends of which are slidably received within the second channel to move the second clip arms between an open configuration, in which distal ends of the second clip arms are separated from one another to receive the tissue therebetween, and a closed configuration, in which the distal ends of the second clip arms are drawn toward one another to grip the tissue therebetween. The distal ends of the second clip arms are configured to engage the pivot feature of the first clip so that the first and second clips are releasably and pivotally connected to one another in an insertion configuration.

In an embodiment, the system further includes a first core member received between and connected to the proximal ends of the first pair of clip arms to couple the proximal ends of the first pair of clip arms to one another, the first core member including a locking feature movable between an unlocked configuration, in which the first core member is slidable within the first channel, and a locked configuration, in which the locking feature of the first core member engages a corresponding portion of the first capsule to lock the first pair of clip arms in the closed configuration. In addition, the system further includes a second core member received between and connected to the proximal ends of the second pair of clip arms to couple the proximal ends of the second pair of clip arms to one another, the second core member including a locking feature movable between an unlocked configuration, in which the second core member is slidable within the second channel, and a locked configuration, in which the locking feature of the second core member engages a corresponding portion of the second capsule to lock the second pair of clip arms in the closed configuration.

In an embodiment, the system further includes a control member including an enlarged distal end and a remaining length extending proximally therefrom, wherein, in the insertion configuration, the enlarged distal end is releasably received within a proximal portion of the first core member so that the remaining length passes through an opening extending through the pivot feature of the first clip, between the second clip arms and through the second core member to a proximal end that is configured to be accessible a user, the control member longitudinally movable relative to the first capsule to move the first clip between the open and the closed configuration.

In an embodiment, each of the first and second core members includes an opening extending therethrough in communication with a cavity thereof, the opening configured to deform to permit passage of the enlarged distal end therethrough when subject to a force exceeding a predetermined threshold value.

In an embodiment, each of the first and second core members is connected to a corresponding one of the first and second pair of clip arms via a connector received within holes extending through each of the proximal ends of the first and second clip arms and through an elongated opening extending through each of the first and second core members.

In an embodiment, the connector is slidable from a proximal end of the elongated opening to a distal end of the elongated opening to move the locking feature of a corresponding one of the first and second core members from the unlocked configuration to the locked configuration, the connector configured to interface with a portion of the locking feature of the corresponding one of the first and second core members as the connector is moved distally through the elongated opening thereof.

In an embodiment, the each of the first and second core members is formed of a stamped sheet of metal sized and shaped to be bent into a configuration including a proximal portion defining a cavity therewithin for housing the enlarged distal end of the control member and a distal portion including the locking feature.

In an embodiment, the pivot feature includes a curved distal surface configured to pivotally engage a correspondingly curved portion of the distal ends of the second pair of clip arms.

In an embodiment, the pivot feature may be one of machined into a material of the first capsule, stamped from a portion of a wall of the first capsule, and formed of a separate element connected to the proximal end of the first capsule.

In an embodiment, the pivot feature includes a pair of tabs extending from diametrically opposing portions of the proximal end of the first capsule toward a centerline thereof, a spacing between the pair of tabs configured to permit slidable motion of the remaining length of the control member therebetween while preventing passage of the enlarged distal end therethrough.

In an embodiment, the pair of tabs is configured to break away from the first capsule when subject to a force exceeding a predetermined threshold value.

In an embodiment, the first capsule includes a stop positioned distally of the pivot feature, the stop configured to prevent a proximal movement of the second pair of clip arms relative thereto when the distal ends of the second pair of clip arms is engaged with the pivot feature of the first clip, in the insertion configuration.

In an embodiment, the stop is stamped from a material of the wall of the first capsule and bent radially inwards.

In an embodiment, the distal ends of the second pair of clip arms includes an opening extending between distal tips thereof the opening being sized, shaped and configured to permit passage of the enlarged distal end of the control member therethrough.

In an embodiment, proximal portions of the second pair of claim arms include longitudinal slots extending therethrough and therealong, the longitudinal slots sized, shaped and configured to permit passage of the enlarged distal end of the control member between the second pair of clip arms.

In addition, the present disclosure relates to a method for treating target tissue. The method includes inserting a plurality of clips through a working channel of an endoscope to a target site within a body, the plurality of clips including a first, distal-most clip and a second, immediately adjacent clip, the first clip including a first capsule and a first pair of clip arms, proximal ends of which are slidably received within the first capsule to move the first clip between an open configuration and closed configuration, a proximal end of the first capsule including a pivot feature extending across a first channel of the first capsule, the second clip including a second capsule and a second pair of clip arms, proximal ends of which are slidably received within the second capsule, the second pair of clip arms clipped over the pivot feature of the first clip to releasably and pivotally connect the first and second clips to one another in an insertion configuration; moving the first clip between the open configuration, in which distal ends of the first pair of clip arms are separated from one another to receive tissue therebetween, and the closed configuration, in which the distal ends of the first pair of clip arms are drawn toward one another to grip tissue therebetween, until target tissue is gripped thereby, as desired; locking the first clip in the closed configuration by moving a locking feature of a first core member, via which a control member is coupled to the first pair of arms to move the first pair of clip arms relative to the first capsule, from an unlocked configuration toward a locked configuration in which the locking feature engages a corresponding portion of the first capsule; and initiating a deployment the first clip by drawing the control member proximally until a force exerted on the control member exceeds a predetermined threshold value so that an enlarged distal end of the control member received within a portion of the first core member causes a deformation thereof such that the enlarged distal end of the control member is released from the first core member and drawn proximally between the second pair of clip arms to engage a second core member connecting proximal ends of the of the second pair of clip arms.

In an embodiment, the method further includes moving the control member distally relative to the second capsule so that the second pair of clip arms is moved from a closed configuration toward an open configuration to release the pivot feature of the first clip therefrom, separating the first clip from the second clip.

In an embodiment, during the deployment of the first clip, the enlarged distal end of the control member is drawn proximally against tabs of the pivot feature until a force exerted thereon exceeds a predetermined threshold value, causing the tabs to break away from the first capsule and separating the first clip from the second clip.

In an embodiment, the method further includes moving the second clip between the open configuration, in which distal ends of the second pair of clip arms are separated from one another to receive tissue therebetween, and the closed configuration, in which the distal ends of the second pair of clip arms are drawn toward one another to grip tissue therebetween, until target tissue is gripped thereby, as desired; locking the second clip in the closed configuration by moving a locking feature of the second core member, via which the control member is coupled to the second pair of arms to move the second pair of clip arms relative to the second capsule, from an unlocked configuration toward a locked configuration in which the locking feature engages a corresponding portion of the second capsule; and initiating a deployment the second clip by drawing the control member proximally until a force exerted on the control member exceeds a predetermined threshold value so that the enlarged distal end of the control member received within a portion of the first core member causes a deformation thereof such that the control member is released from the second core member.

In an embodiment, the distal ends of the second pair of clip arms abut against a stop positioned distally of the pivot feature of the first capsule so that the second clip is prevented from moving distally relative to the first clip are the plurality of clips are inserted to a target area.

### BRIEF DESCRIPTION OF THE DRAWINGS

FIG. 1 shows a cross-sectional side view of a clipping system according to an exemplary embodiment of the present disclosure;

FIG. 2 shows a partially transparent side view of the clipping system of FIG. 1, in a pivoted configuration;

FIG. 3 shows an enlarged perspective view of a first and second clips of the clipping system according to FIG. 1;

FIG. 4 shows an enlarged, cross sectional side view of the first and second clips of the clipping system according to FIG. 1;

FIG. 5 shows a perspective view of a pivot feature of the clipping system of FIG. 1, according to another exemplary embodiment;

FIG. 6 shows a side view of a distal end of clip arms of a clip of the clipping system according to FIG. 1;

FIG. 7 shows a perspective view of clip arms of a clip of the clipping system according to FIG. 1;

FIG. 8 shows a perspective view of an exemplary control member of the clipping system according to FIG. 1;

FIG. 9 shows a partially cross-sectional side view of a control member extending through a core member of a clip according to the clipping system of FIG. 1;

FIG. 10 shows a partially cross-sectional side view of an enlarged distal end of a control member received within a cavity of a core member according to the clipping system of FIG. 1;

FIG. 11 shows a cross-sectional side view of a portion of a clipping system according to another exemplary embodiment of the present disclosure, with a control member received within a first and second clip of the clipping system;

FIG. 12 shows a cross-section side view of the portion of the clipping system of FIG. 11, with the control member engaging a pivot feature of the first clip during a deployment of the first clip;

FIG. 13 shows a cross-sectional side view of the portion of the clipping system of FIG. 11, with the pivot feature broken away to separate the first clip from the second clip and deploy the first clip;

FIG. 14 shows a side view of a clipping system according to another exemplary embodiment of the present disclosure;

FIG. 15 shows a perspective view of the clipping system according to FIG. 14, each of a plurality of clips clipped about a periphery of a target tissue defect; and

FIG. 16 shows another perspective view of the clipping system according to FIG. 15, a suture coupled to each of the plurality of clips tightened to close the target tissue defect.

### DETAILED DESCRIPTION

The present disclosure may be further understood with reference to the following description and the appended drawings, wherein like elements are referred to with the same reference numerals. The present disclosure is directed to an endoscopic clipping system for treating internal tissue perforations, defects and/or bleeds. Exemplary embodiments of the present disclosure describe a clipping system comprising a plurality of, which may be loaded within a single insertion device so that multiple clips may be deployed during a single pass through an endoscope or other insertion device.

Each of the first and second clips include a pair of clip arms, proximal ends of which are slidable within a capsule to move the clip arms between an open configuration, in which distal ends of the clip arms are separated from one another to receive target tissue therebetween, and a closed configuration, in which the clip arms are drawn into the capsule so that the distal ends of the clip arms are moved toward one another to grip the target tissue therebetween. A proximal end of each capsule includes a pivot feature configured to be received between jaws of an immediately proximal clip in a manner which connects adjacent clips to one another while also permitting the connected clips to pivot relative to one another so that the plurality of clips may be inserted along a tortuous path to a target site within the body (e.g., through a flexible insertion instrument inserted via a natural bodily orifice through a natural body lumen to the target site). In a further embodiment, proximal ends of a proximal-most clip may be configured to couple, via a connector, to a control member extending through the insertion device.

During a deployment of a first clip (a distal-most clip), for example, a second clip immediately proximal to the first clip releases the pivot feature of the first clip from between jaws separating the first clip from the second clip. It will be understood by those of skill in the art that the terms proximal and distal, as used herein, are intended to refer to a direction toward (proximal) and away from (distal), respectively, a user of the system.

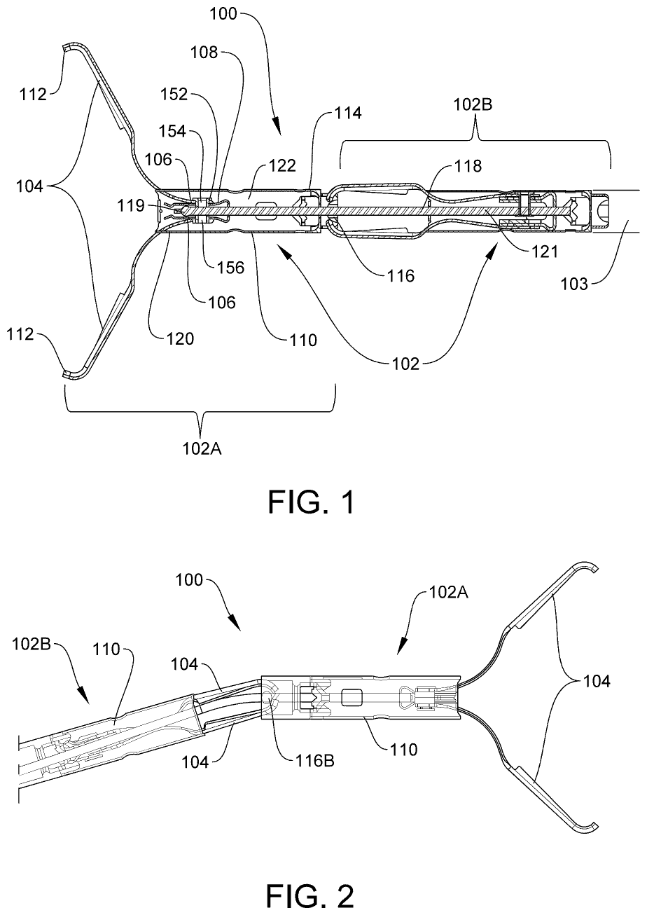

FIGS. 1-10 show a clipping system 100 according to an exemplary embodiment for treating tissue defects comprising a plurality of clips 102 coupled to one another and configured to be inserted through a working channel of a flexible endoscope or other suitable insertion device, to a target site within a body adjacent to tissue to be treated by a first one of the clips 102. As shown in FIG. 1, each of the clips 102 includes a pair of clip arms 104, proximal ends 106 of which are coupled to one another via a core member 108 slidably received within a capsule 110 so that each of the clips 102 may be moved between an open configuration, in which distal ends 112 of the clip arms 104 are separated from one another to receive tissue therebetween and a closed configuration, in which the distal ends 112 of the clip arms 104 are drawn toward one another to grip tissue. A proximal end 114 of the capsule 110 of each of the clips 102 includes a pivot feature 116 configured to be received between clip arms 104 of an immediately proximal clip 102 so that adjacent clips 102 are connected to one another in a clip chain while also being permitted to pivot relative to one another, as shown in FIG. 2.

In an exemplary embodiment, the pivot feature 116 of a first clip 102A of the clips 102 is received between the clip arms 104 of a second clip 102B immediately proximal to the first clip 102A so that the first and second clips 102A, 102B are pivotally connected to one another as shown in FIG. 2. Thus, the chain of clips 102 may be navigated through even tortuous paths of a body lumen to a target site within the body. Although the exemplary embodiments show and describe two clips 102—the first clip 102A and the second clip 102B—it will be understood by those of skill in the art that the chain of clips 102 of the clipping system 100 may include any number of clips 102 while remaining flexible enough to navigate through tortuous paths of a body lumen. For example, in a further embodiment, the clipping system 100 may include ten clips 102. As will be described in further detail below, each of the clips 102 may be moved between the open and closed configurations, and subsequently deployed, via a control member 118 including an enlarged distal end 119 configured to releasably engage the core member 108 of a distal-most one of the clips 102.

As described above, each of the clips 102 includes a capsule 110 and clip arms 104 movable relative to the capsule 110 so that the clip arms 104 move between the open and closed configurations as they are retracted into and extended distally out of the capsule 110. According to an exemplary embodiment, the capsule 110 extends from the proximal end 114 to a distal end 120 and includes a channel 122 extending therethrough. As indicated above, the proximal end 114 includes a pivot feature 116 extending across the channel 122 of the capsule 110 with an opening 124 extending through the pivot feature 116 and configured so that the control member 118 can pass slidably therethrough.

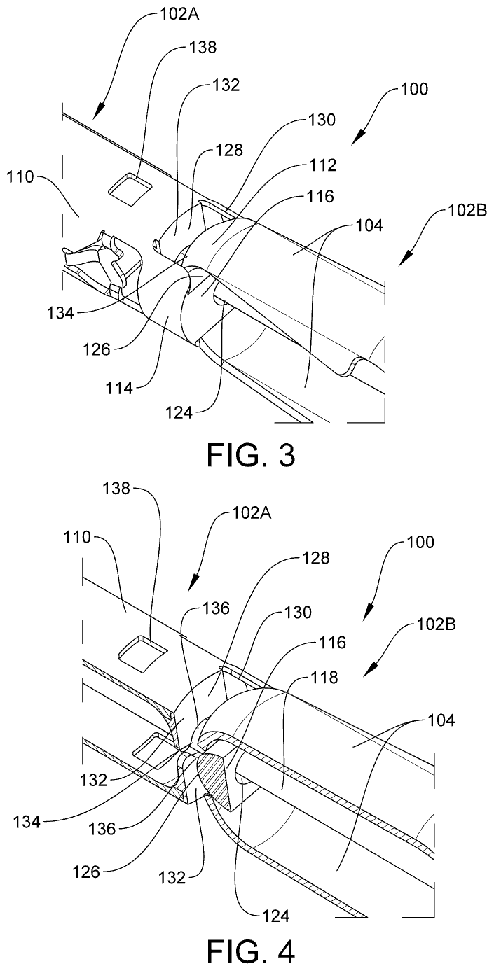

As shown in FIGS. 3-5, the pivot feature 116 includes a curved distal surface 126 configured to engage clip arms 104 of the immediately adjacent clip to form a hinge joint therebetween. The opening 124 extends through the pivot feature 116 coaxially relative to an axis of the channel 122 of the capsule 110 so that a length of the control member 118 may pass through the channel 122 and the pivot feature 116 of the capsules 110 of each of the clips 102.

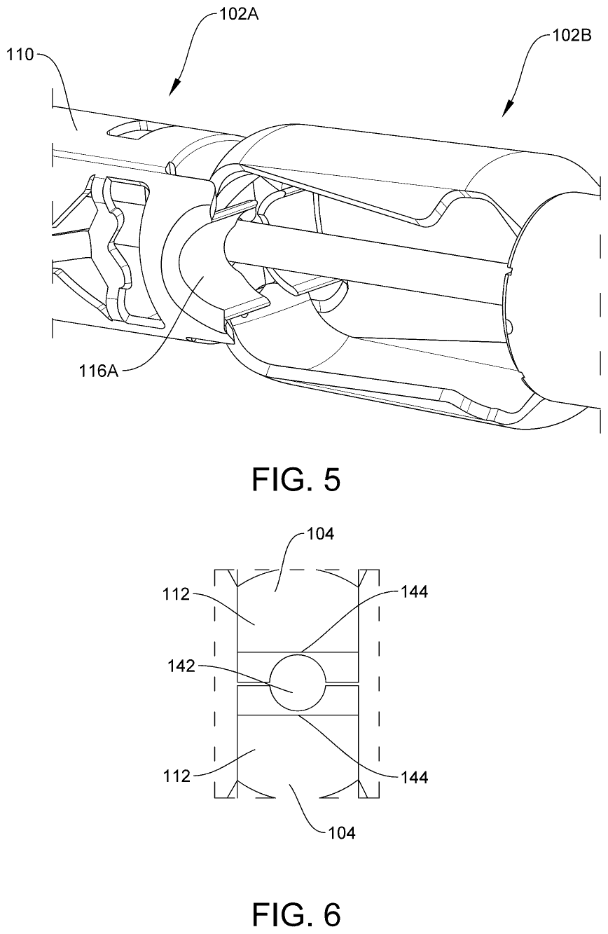

In an exemplary embodiment, as shown in FIGS. 3-4, the pivot feature 116 is machined out of a portion of the capsule 110 although those skilled in the art will understand that the pivot feature 116 may be formed in any of a variety of suitable manners. In another exemplary embodiment, as shown in FIG. 5, a pivot feature 116A, is stamped from a portion of the wall 130 of the capsule 110 and bent radially inward to include all of the features of the pivot feature 116 (e.g., the curved distal surface 126 and the opening 124), as described above. In yet another embodiment, as shown in FIG. 2, a pivot feature 116B is formed as a separate piece connected to the capsule 110 via, for example, crimp or weld. It will be understood by those of skill in the art, however, that the pivot feature 116 may be formed via any of a variety of configurations so long as the pivot feature 116 of each of the clips 102 is configured to be pivotally coupled to the clip arms 104 of an adjacent clip 102, as described above.

The capsule 110 of each of the clips 102 also includes a stop 128 positioned proximate the proximal end 114 of the capsule 110, distally of the pivot feature 116 so that clip arms 104 of an immediately proximal clip 102 that grip that pivot feature 116 are prevented from moving distally relative to the capsule 110 of the clip 102 to which the immediately proximal clip 102 is connected. The stop 128, however, also includes an opening 134 through which a portion of a length of the control member 118 may be slidably received.

In an exemplary embodiment, the stop 128 is formed via a portion 132 of a wall 130 of the capsule 110 that is stamped and bent radially inward. In an exemplary embodiment, the stop 128 may be formed via two stamped portions diametrically opposed to one another. The opening 134 is formed via a space extending between radially innermost edges 136 of the stamped portions 132 that are bent inward is sized, shaped and configured to receive a portion of the control member 118 therebetween. However, those skilled in the art will understand that the stop 128 may be formed in any of a variety of suitable manners without departing from the scope of the disclosed embodiments.

In an exemplary embodiment, when the pivot feature 116 of the first clip 102A is received and clipped between the distal ends 112 of the clip arms 104 of the second clip 102B, the distal ends 112 of the clip arms 104 of the second clip 102B pivotally engage the pivot feature 116 of the first clip 102A so that the second clip 102B may be pivoted relative to the first clip 102A, about the pivot feature 116, as shown in FIG. 2. In addition, as shown in FIGS. 3-5, the distal ends 112 of the clip arms 104 of the second clip 102B abut the stop 128 of the first clip 102A to prevent the second clip 102B from moving further distally with respect to the capsule 110 of the first clip 102A. As shown in FIGS. 3-5, the distal ends 112 of the clip arms 104 of the second clip 102B is received between the stop 128 and the pivot feature 116 of the first clip 102A.

The capsule 110 of each of the clips 102 additionally includes one or more locking structures 138 each being configured to engage a corresponding locking feature 140 of the core member 108, which will be described in further detail below. According to an exemplary embodiment, the locking structures 138 are formed in the wall 130 of the capsule 110 and, in a particular embodiment, are configured as windows extending laterally through the wall 130. Alternatively, the locking structures may be formed as indentations or other walls within the capsule 110 that are configured to engage the corresponding locking feature in a manner similar to the engagement between the locking features 140 and the edges of the locking structures 138.

Each of the clip arms 104 extends from its proximal end 106 to its distal end 112. As described above, the proximal ends 106 of the clip arms 104 are slidably received within the channel 122 so that the clip arms 104 are movable relative to the capsule 110 between the open and the closed configurations via manipulation of the control member 118, as will be described in further detail below. According to an exemplary embodiment, the clip arms 104 are biased toward the open configuration so that, when the clip arms 104 are advanced distally out of the capsule 110, the distal ends 112 of the clip arms 104 move apart from one another toward the open configuration under their natural bias. When the clip arms 104 are drawn proximally into the capsule 110, the clip arms 104 are constrained by the wall of the capsule 110 and drawn together toward the closed configuration, with the distal ends 112 adjacent to one another. Those skilled in the art will understand that a number of other mechanisms for opening and closing the clip arms 104 may be employed.

In an exemplary embodiment, distal ends 112 of each of the clip arms 104 are curved inward, toward a centerline of the clip 102, so that the distal ends 112 are sized, shaped and configured to extend about the curved distal surface 126 of the pivot feature 116 of an adjacent clip 102 to facilitate pivoting of adjacent ones of the clips 102 relative to one another. The distal ends 112 are also shaped such that, when the clip arms 104 are drawn together in the closed configuration, an opening 142 is formed between the distal tips 144 of the distal ends 112, as shown in FIG. 6. As will be described in further detail below, the opening 142 is sized, shaped and configured to permit passage of a portion of the control member 118 therethrough.

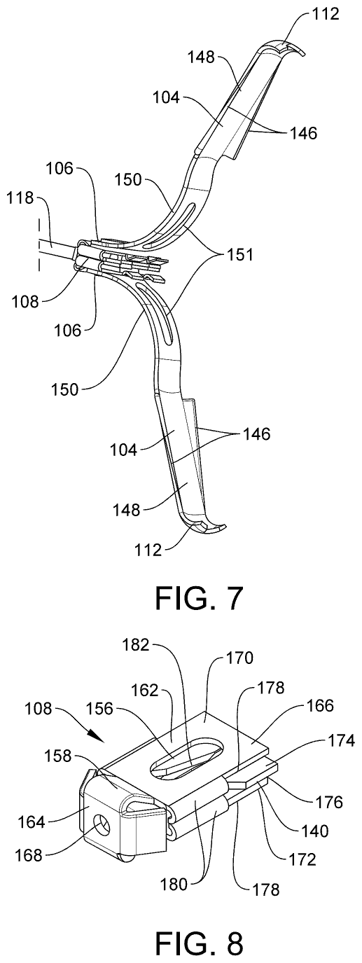

According to an exemplary embodiment, as shown in FIG. 7, each of the clip arms 104 may also include engaging features 146 along a distal portion 148 thereof. The engaging features 146 of this embodiment are configured to engage the distal end 120 of the capsule 110 to prevent the clip arms 104 from being moved proximally thereinto beyond a desired maximally proximal point. In an exemplary embodiment, the engaging features 146 extend along distal portions 148 of the clip arms 104, laterally outward therefrom, so that the distal portions 148 of the clip arms 104 have a width greater than proximal portions 150 of the clip arms 104, which are sized to permit them to be drawn proximally into the capsule 110. In one example, the engaging features 146 are configured as wings extending laterally from longitudinal edges of distal portions 148 of the clip arms 104.

The proximal portions 150 of the clip arms 104 may include longitudinal slots 151 extending therethrough and along a portion of a length thereof. The longitudinal slots 151 are sized, shaped, and configured to provide sufficient spacing between the proximal portions 150 of the clip arms 104 (which in the closed configuration may be bent toward one another) through which a portion of the control member 118 may be drawn proximally during deployment of a distal-most one of the clips 102 (e.g., the first clips 102A). For example, upon separation of an enlarged distal end 119 of the control member 118 from the core member 108 of the first clip 102A, the control member 118 is drawn proximally relative to the chain of clips 102 until the enlarged distal end 119 is drawn between the clip arms 104 of the second clip 102B to engage the core member 108 of the second clip 102B, as will be described in further detail below.

As described above, each of the proximal ends 106 of the clip arms 104 may be connected to the core member 108. In an exemplary embodiment, each of the proximal ends 106 of the clip arms 104 may include a hole 152 extending therethrough so that, when the clip arms 104 are diametrically opposed relative to one another, the holes 152 of the clip arms 104 are aligned with one another so that a connector such as, for example, a rivet 154 may be passed through the holes 152 at the proximal ends 106 of the clip arms 104 and through a corresponding opening 156 of the core member 108 to couple the clip arms 104 to the core member 108. The holes 152 of the clip arms 104 are positioned along the clip arms 104 proximally of the longitudinal slots 151.

Each core member 108, as shown in FIGS. 8-10, is sized and configured to be longitudinally slidable within the channel 122 of the corresponding capsule 110. According to an exemplary embodiment, each core member 108 extends longitudinally from a proximal end 164 to a distal end 166 and comprises a proximal portion 158 including a cavity 160 configured to releasably receive the enlarged distal end 119 of the core member 108 and a distal portion 162 configured to be connected to the proximal ends 106 of the clip arms 104. The proximal end 164, which defines a proximal surface of the cavity 160, includes an opening 168 extending therethrough, along a central axis aligned with a longitudinal axis of the core member 108. The opening 168 is open to and in communication with the cavity 160 so that a remaining length 121 of the control member 118, which extends proximally from the enlarged distal end 119, extends proximally therethrough, when the enlarged distal end 119 is received within the cavity 160.

In an exemplary embodiment, the opening 168 is sized and shaped so that the enlarged distal end 119 is prevented from passing proximally therethrough as the control member 118 is extended distally and drawn proximally to open and close the clip 102. A portion of the core member 108 surrounding the opening 168, however, is formed of a material configured to deform and/or fail when subject to a force exceeding a predetermined threshold value. Thus, as described in further detail below, when the clip 102 is locked and continued proximally directed force is applied to the control member 118, the enlarged distal end 119 exerts an increasing proximally directed force on the opening 168 as further proximal motion of the control member 118 is prevented. When this force exerted on the opening 168 exceeds the predetermined threshold value, the opening 168 deforms to permit the enlarged distal end 119 to pass proximally therethrough, deploying the clip 102 and permitting the enlarged distal end 119 pass proximally through the stop 128 and through the opening 124 in the pivot feature 116 of the capsule 110 of the distal-most clip 102A to enter the capsule 110 of the immediately proximal second clip 102B.

The distal portion 162 includes the opening 156 via which the core member 108 is configured to be coupled to the clip arms 104 via the rivet 154. In an exemplary embodiment, the opening 156 is elongated along a length of the distal portion 162 so that the rivet 154 is slidable along a length thereof from a proximal position within the opening 156 toward a distal position within the opening 156 to move locking features 140 thereof toward a locking configuration, as will be described in further detail below.

In an exemplary embodiment, the core member 108 is formed of a stamped sheet of material (e.g., metal) that is sized and shaped so that, when bent, will include the proximal and distal portions 158, 162, as described above. In one embodiment, the material is bent about the proximal end 164 to further include a first surface 170 and a second surface 172. A portion of the first and second surfaces 170, 172 extending along the proximal portion 158 (along with the proximal end 164) defines the cavity 160 therebetween. A portion of the first and second surfaces 170, 172 extending along the distal portion 162 of the core member 108 is, in one embodiment, substantially planar, extending parallel to one another and to a longitudinal axis of the capsule 110 within which the core member 108 is received.

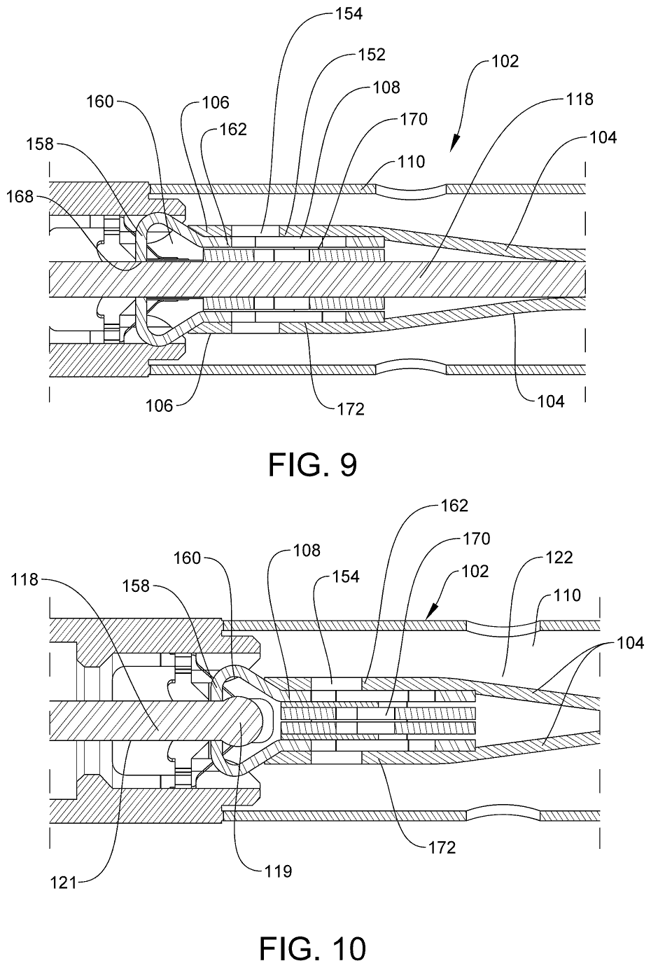

In an exemplary embodiment, the first and second surfaces 170, 172 are biased toward a collapsed configuration in which the first and second surfaces 170, 172 are biased toward one another. As shown in FIG. 9, the core member 108 is in an expanded configuration such that the first and second surfaces 170, 172 along the distal portion 162 are configured to be moved apart from one another to accommodate a portion of the remaining length 121 of the control member 118 therebetween. Once the control member 118 has been removed from between the first and second surfaces 170, 172 along the distal portion 162, however, the first and second surfaces 170, 172 revert toward their biased collapsed configuration, as shown in FIG. 10, by moving toward one another.

As will be understood by those of skill in the art, the opening 156 of the distal portion 162 extends along a central axis to extending through each of the first and second surfaces 170, 172 and, as described above, is elongated along the length of the distal portion 162. The rivet 154, which is received within the opening 156, extends along a central axis aligned with and/or parallel to the central axis of the opening 156 as it is movable along a length of the opening 156. The rivet 154 includes a hole (not shown) extending therethrough along an axis transverse to the central axis thereof to slidably receive a portion of the remaining length 121 of the control member 118 therethrough. In an exemplary embodiment, the axis along which the hole of the rivet 154 extends is substantially perpendicular to the central axis of the rivet 154.

In an exemplary embodiment, portions of the material of the core member 108 extending from the first and second surfaces 170, 172 are also bent inward to extend between the first and second surfaces 170, 172 to define the locking features 140. In an exemplary embodiment, the locking features 140 are configured as a pair of wings 174 extending between the first and second surfaces 170, 172 and movable between an unlocked configuration and the locked configuration. In the unlocked configuration, exterior longitudinal edges 176 of the wings 174 are substantially aligned with the longitudinal edges 178 of the first and second surfaces 170, 172 so that the core member 108 is slidable longitudinally within the channel 122 of the capsule 110 to move the clip arms 104 between the open and closed configurations. In the locked configuration, a portion of each of the wings 174 is moved radially outward, beyond longitudinal edges 178 of the first and second surfaces 170, 172, to engage the locking structures 138 of the capsule 110.

Each of the of the wings 174 may be connected to either the first surface 170 or the second surface 172 via, for example, a bent portion 180 extending along longitudinal edges 178 of one the first and second surfaces 170, 172. This bent portion 180 may be deformed as the core member 108 is moved from the unlocked configuration to the locked configuration, in which a distal portion of the wings 174 is moved radially outward.

In an exemplary embodiment, each of the wings 174 may be further defined via interior longitudinal edges 182 angled so that a portion of each of the wings 174 overlaps with the elongated opening 156. In particular, a portion of each of the wings 174 overlaps the elongated opening 156 so as not to interfere with the rivet 154, when the rivet 154 is received in the proximal position within the elongated opening 156 in the unlocked configuration. It will be understood by those of skill in the art that the interior longitudinal edges 182 which overlap with the elongated opening 156 may hold the rivet 154 in the proximal position therewithin as the clip arms 104 are moved between the open and the closed configurations.

When it is desired to lock the clip 102, however, the rivet 154 engages the angled interior longitudinal edges 182 of the wings 174, as it is moved from the proximal position to the distal position within the elongated opening 156, to exert a force thereon. When a force exerted on the interior longitudinal edges 182 via the rivet 154 exceeds a predetermined threshold value, it deforms the core member 108 permitting the wings 174 to move from the unlocked configuration toward the locked configuration.

Each of the clips 102 is assembled so that the core member 108 is received between the proximal ends 106 of the clip arms 104. The elongated opening 156 of the core member 108 is aligned with the holes 152 of the extending through the proximal ends 106 of the clip arms 104. The rivet 154 extends through the holes 152 of the clip arms 104 and through the proximal portion of the elongated opening 156 of the core member 108. The core member 108, assembled with the clip arms 104, is slidably received within the channel 122 of the capsule 110.

In an initial insertion configuration, each of the clips 102 in the chain of clips 102 is in the closed configuration. The clips 102 are connected to one another so that they may be loaded in a working channel of an endoscope or other insertion device. In particular, the first clip 102A is positioned distally of the second clip 102B so that the clip arms 104 of the second clip 102B receives the pivot feature 116 of the first clip 102A between the clip arms 104 of the second clip 102B. The distal ends 112 of the second clip 102B engage the pivot feature 116 of the first clip 102A to both connect the first and second clips 102A, 102B and to permit the first and second clips 102A, 102B to pivot relative to one another. Since adjacent clips 102 are able to pivot relative to one another, the chain of clips 102 may be inserted through even tortuous paths of a body lumen to a target area. It will be understood by those of skill in the art that a proximal-most one of the chain of clips 102 may be releasably coupled to, for example, a catheter 103, to facilitate to insertion of the chain of clips 102, through the endoscope or other insertion device, to the target area within the body.

In an initial configuration, the enlarged distal end 119 of the control member 118 is received within the cavity 160 of the core member 108 of the first clip 102A. The remaining length 121 of the control member 118 passes proximally through the opening 156 of the core member 108 of the first clip 102A, between the clip arms 104 and through the core member 108 of the second clip 102B (and similarly through all of the clips 102 in the chain of clips 102 proximal of the second clip 102B) to a proximal end of the system such that it is accessible to a user via, for example, a control mechanism or actuator at a proximal end of the insertion device.

It will be understood by those of skill in the art that although all of the clips 102 are shown and described as having the same features, in some embodiments, one or more of the clips 102 may have differing features depending on, for example, a position of the clip along the chain of clips 102. For example, while each of the clips 102 is described as including longitudinal slots 151 along the clip arms 104 and an opening 142 formed via distal tips 144 of the clip arms 104, the distal-most one of the clips 102 (e.g., the first clip 102A) is not required to include these features as the enlarged distal end 119 does not pass between distal portions of the clip arms 104 of the first clip 102A since the enlarged distal end 119 of the control member 118 is positioned proximate the proximal end 106 of the clip arms 104 of the first clip 102A in the initial insertion configuration. In another example, a proximal-most one of the clips 102 need not include a pivot feature 116 or a stop 128 as the proximal-most clip 102 is not coupled to a more proximal clip 102 and may be coupled to the catheter 103 in any desired manner.

According to an exemplary method utilizing the clipping system 100, the chain of clips 102 is inserted through, for example, a working channel of an endoscope to a target site within a body. The chain of clips 102 is inserted to the target site in the initial insertion configuration, in which the clips 102 in the chain are connected to one another via the hinge joints formed via the interaction of the pivot features 116 and the clip arms 104, so that the chain of clips 102 may be navigated through even tortuous paths within body lumens to the target site. As described above, a proximal-most one of the clips 102 may be releasably coupled to the catheter 103 to facilitate insertion of the chain of clips 102 through the endoscope or other insertion device.

Once the chain of clips 102 has reached the target site, the user advances the control member 118 distally relative to the capsules 110 of the clips 102. As described above, in the initial insertion configuration, the enlarged distal end 119 of the control member is received within the core member 108 of the distal-most clip 102 (i.e., the first clip 102A) so that the distal motion of the control member 118 relative to the capsules 110 moves the clip arms 104 of the first clip 102A distally out of the capsule 110 of the first clip 102A toward the open configuration, under their natural bias, to receive between the distal ends 112 thereof.

When the target site is reached, the user positions the first clip 102A so that the distal ends of the open clip arms 104 contact desired portions of tissue (e.g., portions of tissue on opposite sides of an opening in the tissue that is to be closed), The user then draws the control member 118 proximally (or advances the catheter 103 distally over the control member 118) so that, as the clip arms 104 of the first clip 102A are drawn into the capsule 110 thereof, the clip arms 104 are drawn toward one another to draw together and grip the tissue contacted by the distal ends 112 of the clip arms 104. The first clip 102A may be moved between the open and closed configurations, as desired, until a target portion of the tissue has been gripped between the clip arms 104 as desired (e.g., when opposite sides of an opening in the tissue have been drawn together to close the opening).

When the user is satisfied that the first clip 102A is in a desired position gripping the target tissue, the user may finally deploy the first clip 102A by locking the clip arms 104 within the capsule 110 in the closed configuration and releasing the first clip 102A from the immediately proximal second clip 102B. In particular, according to an exemplary embodiment, when the first clip 102A is gripping the target tissue, as desired, the user draws the control member 118 proximally until engaging features 146 along the distal portion 148 of the clip arms 104 engage the distal end 120 of the capsule 110, preventing further proximal motion of the clip arms 104 of the first clip 102A relative to the capsule 110 thereof. After this point, continued proximal motion of the control member 118 applies increasing tension to the control member 118 and the core member 108 of the first clip 102A. This draws the core member 108 proximally over the rivet 154 which connects the clip arms 104 to the core member 108 so that the rivet 154 moves from its proximal position within the elongated opening 156 of the core member 108 to a distal position within the elongated opening 156.

As the rivet 154 slides toward the distal portion of the elongated openings 156, the rivet 154 engages portions of the wings 174 overlapping the elongated openings 156. The rivet 154 slides distally along the angled interior longitudinal edges 182 of the wings 174 pushing the wings 174 from the unlocked configuration radially outward toward the locked configuration. In the locked configuration, the wings 174 are moved radially outward relative to the longitudinal axis of the core member 108 to engage the locking structures 138. In an exemplary embodiment, the wings 174 move radially outward to extend into and/or through locking structures 138 configured, in this embodiment, as windows extending through the wall 130 of the capsule 110.

Once the first clip 102A is locked in the clipped/closed configuration—with the core member 108 thereof locked with respect to the capsule 110—the user continues to draw the control member 118 proximally until a force exerted on the opening 168 of the core member 108 of the first clip 102A via the enlarged distal end 119 exceeds the predetermined threshold value, causing the opening 168 to deform to allow proximal passage of the enlarged distal end 119 therethrough. The control member 118 continues to move proximally until the enlarged distal end 119 is received within the cavity 160 of the core member 108 of the immediately proximal clip 102—i.e., the second clip 102B. The control member 118 may now be used to control movement of the second clip 102B between the open and the closed configurations in the same manner described above in regard to the first clip 102A.

In other words, the core member 108 of the second clip 102B remains in the expanded configuration with the first and second surfaces 170, 172 separated from one another by the control member 118 until the enlarged distal end 119 passes between the first and second surfaces 170, 172 to be received within the cavity 160 of the core member 108, thus reverting the core member 108 into the biased collapsed configuration. When the core member 108 reverts into the biased collapsed configuration, a distally directed force applied to the control member 118 exerts a distal force on the core member 108 to thus move the second clip 102B between the open and the closed configurations.

To finally deploy the first clip 102A, the control member 118 is moved distally to move the clip arms 104 of the second clip 102B toward the open configuration until the pivot feature 116 of the first clip 102A is released from therebetween. The second clip 102B may then be clipped over the target tissue, as desired, in a manner substantially similar to that described above in regard to the first clip 102A. While the exemplary embodiments show and describe two clips 102, it will be understood by those of skill in the art that the clipping system 100 may include any number of clips 102 so that the user may continue to place additional clips 102 as desired without removing or reloading the insertion device.

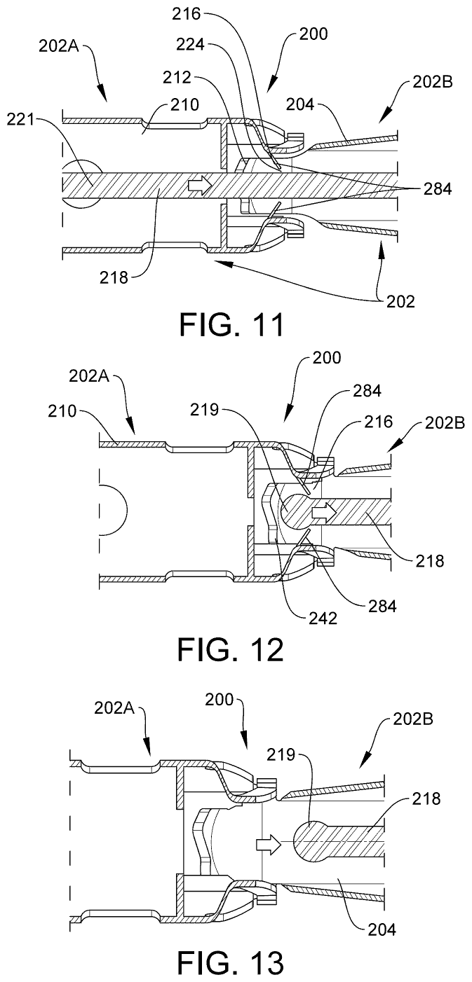

Except as described below, a further embodiment of a clipping system 200 as shown in FIGS. 11-13 is substantially similar to the clipping system 100 described above with a chain of clips 202—e.g., a first clip 202A and a second clip 202B-releasably connected to one another via a pivot feature 216 of at least the first clip 202A so that multiple clips may be deployed to treat a tissue defect during a single pass through a working channel of an endoscope or other insertion device. Each of the clips 202 is substantially similar to the clips 102 described above with respect to the clipping system 100, except that a pivot feature 216 at a proximal end 214 of a capsule 210 of each of the clips 202, is formed via a pair of tabs 284 extending from the proximal end 214 of toward a centerline of the capsule 210. The tabs 284 of each pair are diametrically opposed to one another so that they are received between distal ends 212 of closed clip arms 204 of an adjacent one of the clips 202.

In an exemplary embodiment, the tabs 284 of the first clip 202A are received between the clip arms 204 of the second clip 202B so that the pair of tabs 284 together form an axis about which the second clip 204B may pivot relative to the first clip 202A. A space 224 between the pair of tabs 284 is sized to permit a portion of a length 221 of the control member 218 extending proximally from an enlarged distal end 219 of the control member 218 to be slidably received therebetween, while preventing a passage of the enlarged distal end 219 therethrough. In an exemplary embodiment, a portion of the length 221 of the control member 218 is received between the tabs 284 of the first clip 202A and between the clip arms 204 of the second clip 202B via, for example, an opening 242 formed between the distal ends 212 of the clip arms 204 coaxially relative to a longitudinal axis of each of the clips 202.

Similarly to the clipping system 100, the control member 218 may be moved longitudinally relative to the capsules 210 of the clips 202 to move the clip arms 204 of the first clip 202A between the open and closed configurations, until target tissue is gripped therebetween, as desired. Although not shown, the clip arms 204 of the first clip 202A (and any subsequently deployed clips 202) are movable between the open and closed configurations via a core member which connects proximal ends of the clip arms 204 to the enlarged distal end 219 of the control member 218. Once the target tissue has been gripped between the distal ends 212 of the first clip 202A, as desired, the control member 218 is drawn proximally relative to the capsule 210 until a locking feature of the core member locks the first clip 202A in the closed configuration in a manner substantially similar to that described above for the clips 102.

Upon locking the first clip 202A, deployment is initiated by drawing the control member 218 proximally relative to the first clip 202A, as shown in FIG. 11, until the enlarged distal end 219 is released from the core member of the first clip 202A substantially as described above with respect to clipping system 100. The user continues to move the control member 218 proximally until the enlarged distal end 219 is drawn through the opening 242 between the distal ends 212 of the second clip 202B and drawn against the tabs 284 of the first clip, as shown in FIG. 12. When a force exerted on the tabs 284 via the enlarged distal end 219 exceeds a predetermined threshold value, the tabs 284 bend out of the way or break away from the capsule 210, releasing the first clip 202A from the second clip 202B and allowing the enlarged distal end 219 to move proximally relative to the second clip 202B, as shown in FIG. 13, until the enlarged distal end 219 engages the core member of the second clip 202B to facilitate a subsequent clipping of the second clip 202B.

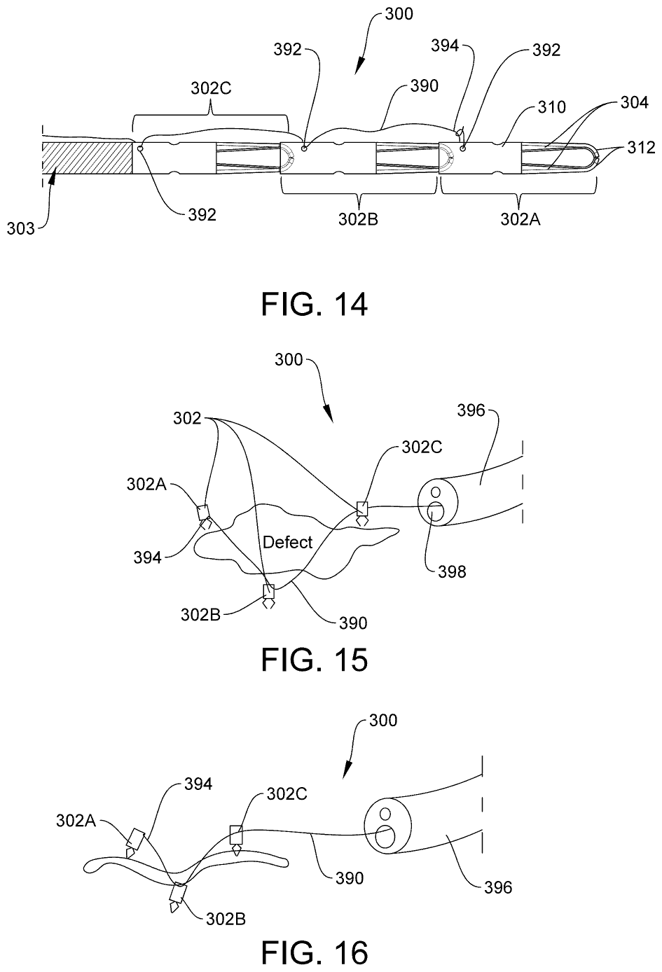

Except as described below, a further embodiment of a clipping system 300 as shown in FIGS. 14-16 is substantially similar to the clipping systems 100, 200 described above, comprising a chain of clips 302 (e.g., a first clip 302A, a second clip 302B, and a third clip 302C) connected to one another via, for example, a pivot feature (not shown) of each of the clips 302. The clips 302 and the pivot features may be substantially similar to any of the clips 102, 202 and the pivot features 116, 216 described above with respect to the clipping systems 100, 200. The clipping system 300, however, further comprises a suture 390 extending through portions of each of the clips 302 so that, upon placement and deployment of each of the clips 302, e.g., about a periphery of a tissue defect, the suture 390 may be tensioned to draw the clips 302 toward one another to close the tissue defect.

Similarly to the clips 102, 202, each of the clips 302 includes a pair of clip arms 304, proximal ends of which are slidably received within a capsule 310 to move the clip arms 304 between an open configuration, in which distal end 312 of the clip arms 304 are separated from one another to receive tissue between the clip arms 304, and a closed configuration, in which the distal ends 312 of the clip arms 304 are drawn toward one another to grip the tissue therebetween. Each of the capsules 310 in this embodiment, however, includes a hole 392 extending through a wall thereof. Each of these holes in one embodiment is configured to receive a portion of the suture 390 therein.

In an exemplary embodiment, the suture 390 extends from a distal end 394 to a proximal end (not shown). The distal end 394 is received within and connected to the hole 392 of a distal-most one of the clips 302 (e.g., the first clip 302A) via, for example, a knot, cinch or other attachment mechanism to fixedly attach the distal end 394 to the distal-most clip 302. A remaining length of the suture 390 passes slidably through the holes 392 of the remaining clips 302 (e.g., the second clip 302B and the third clip 302C) and through a working channel 398 of an endoscope 396 or other insertion instrument, to the proximal end (not shown) which is accessible to a user via, for example, a control handle or actuator at the proximal end of the endoscope/insertion device. It will be understood by those of skill in the art that although the clipping system 300 is shown and described as including three clips 302, the clipping system 300 may include any of a number of a plurality of clips 302.

According to an exemplary method of use for the clipping system 300, the chain of clips 302 is inserted through the endoscope 396 or other insertion device to a target area within a body via for example, a catheter 303 releasably coupled to a proximal-most one of the clips 302 (e.g., 302C). The clips 302 may then be clipped over a portion of target tissue in a manner substantially similar to that described in regard to the clips 102, 202 above. In this embodiment, the clips 302 may be clipped, for example, to tissue at locations spread around a periphery of a target tissue defect to be treated, as shown in FIG. 15. When all of the clips 302 have been placed as desired, tension may be applied to the suture 390 to draw all of the clips 302 and the portions of tissue to which they are clipped toward one another to close and seal the tissue defect.

Since, in an exemplary embodiment, the distal end 394 of the suture is fixedly attached to the first clip 302A, applying tension along the suture 390 draws the suture through the holes 392 of the remaining clips (e.g., the second clip 302B, the third clip 302C) pulling the clips 302 toward one another. As the clips 302 are drawn toward one another, as shown in FIG. 16, edges of the tissue defect are also drawn toward one another to close the tissue defect. Once the tissue defect has been closed, as desired, a cinch or other similar device may be slid along the suture 390 until it abuts the capsule 310 of a last-deployed one of the clips 302 (e.g., third clip 302C) to maintain the tension along a portion of the suture 390 extending between the clips 302 to hold the tissue defect closed. An excess portion of suture extending proximally from the cinch or other device may then be cut, as will be understood by those of skill in the art, so that a remaining portion of the clipping system 300 may be removed from the target area.

It will be apparent to those skilled in the art that various modifications may be made in the present disclosure, without departing from the scope of the disclosure. Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

1-15. (canceled)

16. A clipping system for treating tissue, comprising:
a first clip including a first capsule extending longitudinally from a proximal end to a distal end and including a first channel extending therethrough, and a first pair of clip arms, proximal ends of which are slidably received within the first channel to move the first clip arms between an open configuration, in which distal ends of the first clip arms are separated from one another to receive a tissue therebetween, and a closed configuration, in which the distal ends of the first clip arms are drawn toward one another to grip a tissue therebetween, the proximal end of the first capsule including a pivot feature extending across the first channel thereof; and
a second clip including a second capsule extending longitudinally from a proximal end to a distal end and including a second channel extending therethrough, and a second pair of clip arms, proximal ends of which are slidably received within the second channel to move the second clip arms between an open configuration, in which distal ends of the second clip arms are separated from one another to receive the tissue therebetween, and a closed configuration, in which the distal ends of the second clip arms are drawn toward one another to grip the tissue therebetween, the distal ends of the second clip arms configured to engage the pivot feature of the first clip so that the first and second clips are releasably and pivotally connected to one another in an insertion configuration.

17. The clipping system of claim 16, further comprising:
a first core member received between and connected to the proximal ends of the first pair of clip arms to couple the proximal ends of the first pair of clip arms to one another, the first core member including a locking feature movable between an unlocked configuration, in which the first core member is slidable within the first channel, and a locked configuration, in which the locking feature of the first core member engages a corresponding portion of the first capsule to lock the first pair of clip arms in the closed configuration; and
a second core member received between and connected to the proximal ends of the second pair of clip arms to couple the proximal ends of the second pair of clip arms to one another, the second core member including a locking feature movable between an unlocked configuration, in which the second core member is slidable within the second channel, and a locked configuration, in which the locking feature of the second core member engages a corresponding portion of the second capsule to lock the second pair of clip arms in the closed configuration.

18. The clipping system of claim 17, further comprising:
a control member including an enlarged distal end and a remaining length extending proximally therefrom, wherein, in the insertion configuration, the enlarged distal end is releasably received within a proximal portion of the first core member so that the remaining length passes through an opening extending through the pivot feature of the first clip, between the second clip arms and through the second core member to a proximal end that is configured to be accessible a user, the control member longitudinally movable relative to the first capsule to move the first clip between the open and the closed configuration.

19. The clipping system of claim 18, wherein each of the first and second core members includes an opening extending therethrough in communication with a cavity thereof, the opening configured to deform to permit passage of the enlarged distal end therethrough when subject to a force exceeding a predetermined threshold value.

20. The clipping system of claim 17, wherein each of the first and second core members is connected to a corresponding one of the first and second pair of clip arms via a connector received within holes extending through each of the proximal ends of the first and second clip arms and through an elongated opening extending through each of the first and second core members.

21. The clipping system of claim 20, wherein the connector is slidable from a proximal end of the elongated opening to a distal end of the elongated opening to move the locking feature of a corresponding one of the first and second core members from the unlocked configuration to the locked configuration, the connector configured to interface with a portion of the locking feature of the corresponding one of the first and second core members as the connector is moved distally through the elongated opening thereof.

22. The clipping system of claim 18, wherein each of the first and second core members is formed of a stamped sheet of metal sized and shaped to be bent into a configuration including a proximal portion defining a cavity therewithin for housing the enlarged distal end of the control member and a distal portion including the locking feature.

23. The clipping system of claim 16, wherein the pivot feature includes a curved distal surface configured to pivotally engage a correspondingly curved portion of the distal ends of the second pair of clip arms.

24. The clipping system of claim 16, wherein the pivot feature may be one of machined into a material of the first capsule, stamped from a portion of a wall of the first capsule, and formed of a separate element connected to the proximal end of the first capsule.

25. The clipping system of claim 18, wherein the pivot feature includes a pair of tabs extending from diametrically opposing portions of the proximal end of the first capsule toward a centerline thereof, a spacing between the pair of tabs configured to permit slidable motion of the remaining length of the control member therebetween while preventing passage of the enlarged distal end therethrough.

26. The clipping system of claim 25, wherein the pair of tabs is configured to break away from the first capsule when subject to a force exceeding a predetermined threshold value.

27. The clipping system of claim 24, wherein the first capsule includes a stop positioned distally of the pivot feature, the stop configured to prevent a proximal movement of the second pair of clip arms relative thereto when the distal ends of the second pair of clip arms is engaged with the pivot feature of the first clip, in the insertion configuration.

28. The clipping system of claim 27, wherein the stop is stamped from a material of the wall of the first capsule and bent radially inwards.

29. The clipping system of claim 18, wherein the distal ends of the second pair of clip arms includes an opening extending between distal tips thereof the opening being sized, shaped and configured to permit passage of the enlarged distal end of the control member therethrough.

30. The clipping system of claim 18, wherein proximal portions of the second pair of claim arms include longitudinal slots extending therethrough and therealong, the longitudinal slots sized, shaped and configured to permit passage of the enlarged distal end of the control member between the second pair of clip arms.

31. A method for treating target tissue, comprising:
inserting a plurality of clips through a working channel of an endoscope to a target site within a body, the plurality of clips including a first, distal-most clip and a second, immediately adjacent clip, the first clip including a first capsule and a first pair of clip arms, proximal ends of which are slidably received within the first capsule to move the first clip between an open configuration and closed configuration, a proximal end of the first capsule including a pivot feature extending across a first channel of the first capsule, the second clip including a second capsule and a second pair of clip arms, proximal ends of which are slidably received within the second capsule, the second pair of clip arms clipped over the pivot feature of the first clip to releasably and pivotally connect the first and second clips to one another in an insertion configuration;
moving the first clip between the open configuration, in which distal ends of the first pair of clip arms are separated from one another to receive tissue therebetween, and the closed configuration, in which the distal ends of the first pair of clip arms are drawn toward one another to grip tissue therebetween, until target tissue is gripped thereby, as desired;
locking the first clip in the closed configuration by moving a locking feature of a first core member, via which a control member is coupled to the first pair of arms to move the first pair of clip arms relative to the first capsule, from an unlocked configuration toward a locked configuration in which the locking feature engages a corresponding portion of the first capsule; and
initiating a deployment the first clip by drawing the control member proximally until a force exerted on the control member exceeds a predetermined threshold value so that an enlarged distal end of the control member received within a portion of the first core member causes a deformation thereof such that the enlarged distal end of the control member is released from the first core member and drawn proximally between the second pair of clip arms to engage a second core member connecting proximal ends of the of the second pair of clip arms.

32. The method of claim 31, further comprising:
moving the control member distally relative to the second capsule so that the second pair of clip arms is moved from a closed configuration toward an open configuration to release the pivot feature of the first clip therefrom, separating the first clip from the second clip.

33. The method of claim 31, wherein, during the deployment of the first clip, the enlarged distal end of the control member is drawn proximally against tabs of the pivot feature until a force exerted thereon exceeds a predetermined threshold value, causing the tabs to break away from the first capsule and separating the first clip from the second clip.

34. The method of claim 31, further comprising:
moving the second clip between the open configuration, in which distal ends of the second pair of clip arms are separated from one another to receive tissue therebetween, and the closed configuration, in which the distal ends of the second pair of clip arms are drawn toward one another to grip tissue therebetween, until target tissue is gripped thereby, as desired;
locking the second clip in the closed configuration by moving a locking feature of the second core member, via which the control member is coupled to the second pair of arms to move the second pair of clip arms relative to the second capsule, from an unlocked configuration toward a locked configuration in which the locking feature engages a corresponding portion of the second capsule; and
initiating a deployment the second clip by drawing the control member proximally until a force exerted on the control member exceeds a predetermined threshold value so that the enlarged distal end of the control member received within a portion of the first core member causes a deformation thereof such that the control member is released from the second core member.

35. The method of claim 31, wherein the distal ends of the second pair of clip arms abut against a stop positioned distally of the pivot feature of the first capsule so that the second clip is prevented from moving distally relative to the first clip are the plurality of clips are inserted to a target area.
